(19) United States
(12) Patent Application Publication
(10) Pub. No.: US 2015/0133565 A1
(43) Pub. Date: May 14, 2015

(54) SUSTAINED RELEASE HYDROGEL-BASED STIMULANT

(71) Applicant: University of New Hampshire, Durham, NH (US)

(72) Inventors: Shaojun Yao, Kittery, ME (US); W. Rudolf Seitz, Kensington, NH (US)

(21) Appl. No.: 14/403,264

(22) PCT Filed: Jun. 6, 2013
(86) PCT No.: PCT/US13/44529

§ 371 Date: Nov. 26, 2014

Related U.S. Application Data
(60) Provisional application No. 61/660,268, filed on Jun. 15, 2012.

(51) Int. Cl. A61K 47/32; A61K 31/122; A61K 31/045

(52) U.S. Cl. CPC A61K 47/32; A61K 31/045; A61K 31/122
USPC 514/690; 514/729

(57) ABSTRACT

The present invention relates to a thermo-responsive hydrogel polymer formulated with one or more active agents selected from the group consisting of menthol and menthone, wherein said active agents are effective in invigorating the user and/or effective in relieving fatigue.

### TECHNICAL FIELD

The present invention relates to a sustained-release invigorating compound which is intended for use in relief of fatigue and to provide invigoration. More particularly, the present invention relates to a sustained-release compound (such as an ointment) which comprises a base, a thermo-responsive hydro-gel substance, and invigorating, anti-fatigue ingredients such as menthol and menthone.

### BACKGROUND ART

Peppermint is regularly available as an essential oil (a concentrated hydrophobic liquid containing volatile aroma compounds from plants) which contains menthol (about 20-48%) and Menthone (about 20-46%). Peppermint oil is frequently used as a food additive or fragrance additive. Prior research has shown that ingredients in peppermint oil exhibit effectiveness in reducing sleepiness (Norrish and Dwyer, Int. J. Psychology (2005) 55(3):291-296), raising alertness (Raudenbush, et al., (2009) Am. J. Psychology, 11(2):245-256) and increasing improved performance on clerical tasks (Barker, et al., Perceptive Motor kills (2003) 93(3 Pt 1):1007-1010). A product of Burt's Bees, Inc, Beeswax Lip Balm, contains peppermint oil which provides refreshing benefits for users. Peppermint ingredients are mixed with a hydrophobic base (e.g., petrolatum, bee's wax, plastibase, paraffin, silicone, Vaseline®). However, a hydrophobic base can only be mixed well with a small amount of menthol and menthone. Otherwise the mixture can't maintain a solid or semi-solid shape. Due to the above limitation, peppermint oil in regular lip balms (or products of similar composition) is provided in very small amounts and, therefore, provides limited refreshing benefit. Hence those products cannot be used for invigorating purposes or to relieve drowsiness (fatigue) symptoms. Moreover those lip balm-type products or ointments do not provide sustained-release of active ingredients.

What is needed is a sustained-release invigorating compound such as a spray, gel or ointment that provides relief from drowsiness and fatigue.

### SUMMARY OF THE INVENTION

There are no known ointment bases which comprise a hydrogel and invigorating compounds. To solve this problem, the present inventors have developed, through extensive empirical research, a composition comprising a hydrogel and active ingredients that creates an enhanced invigorating effect that is not possible with the known prior art formulations. Moreover, there are no known prior art compositions suitable for providing a stimulating, invigorating or anti-fatigue effect that can be applied on the skin, under or inside the nose (i.e., in the nasal cavity) to provide sustained stimulating, invigorating or anti-fatigue benefits to the user.

Therefore, the present inventors have developed a sustained-release invigorating (anti-fatigue) composition (e.g., ointment, balm or spray) which can be applied into the nose or on skin around/under the nose. The sustained-release properties of the present invention are effective to maintain the concentration of active ingredients at a sufficiently high level to supply invigorating ingredients to a user over a long period of time. Further, the present invention provides an enhanced ratio of total invigorating ingredients to total composition and an enhanced ratio of invigorating ingredients as related to each of the invigorating ingredients. Besides a sustained-release benefit, the present invention provides users with an enhanced cooling experience that is caused by a synergistic effect created by the menthol and water contained in the hydrogel composition (matrix) of the present invention.

Hence, the present invention has an object of providing a novel, non-obvious and unpredictable sustained-release invigorating composition for nasal delivery with an enhanced invigorating, stimulating anti-fatigue effect.

### DESCRIPTION OF THE FIGURES

FIG. 1 shows the sustained-release of the composition of the present invention (WākUp™). The composition relieves drowsiness via sustained-release technology. X-axis is time in minutes. Y-axis is release is rate of release as a percent of total weight of the test composition.

FIG. 2 shows the released active ingredient content expressed in terms of weight loss percentage over time.

FIG. 3 shows release distribution of menthone in the sustained-release composition of the present invention. Values in the Y axis represent the percentage of released menthone in time spans of 10 minutes over the total menthone added initially.

FIG. 4 shows data on the release of actives from the hydrogel of the present invention as the result of exposure to body temperature. X-axis is time in minutes. Y-axis is release is rate of release as a percent of total weight of the test composition.

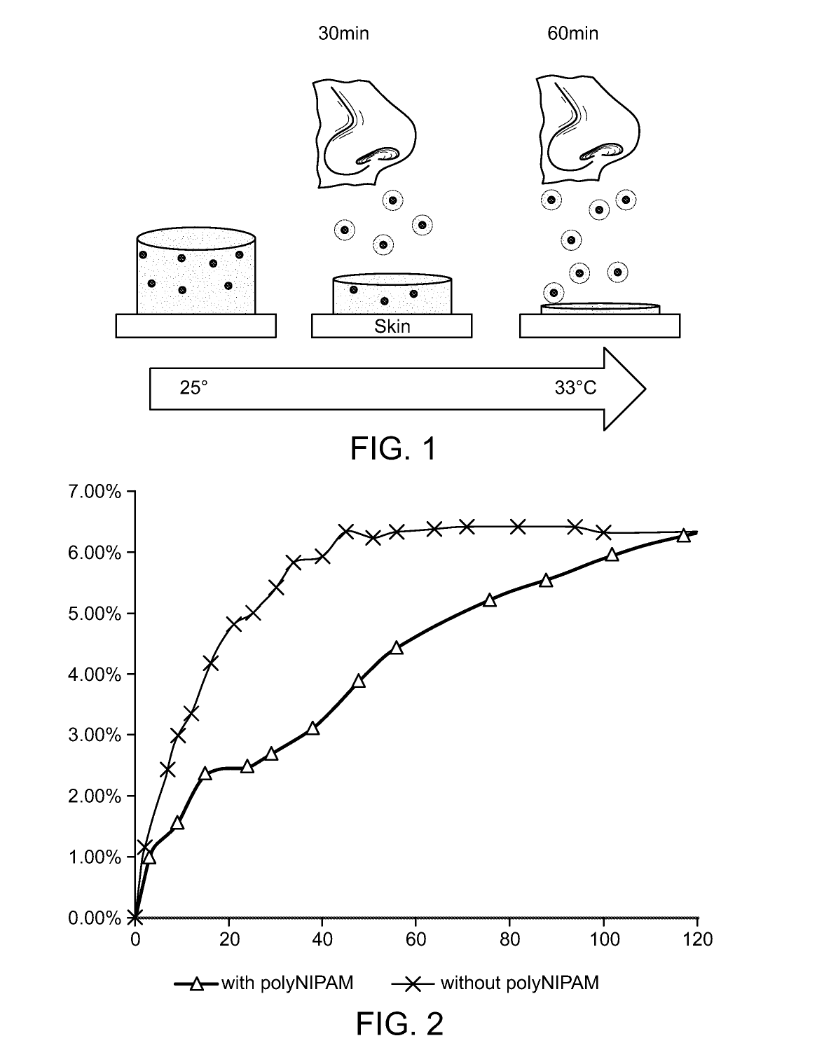

### DETAILED DESCRIPTION OF THE INVENTION

The technology related to the present invention involves use of a thermo-responsive hydrogel. Thermo-responsive hydrogels form swollen states after they absorb water, active ingredients (e.g., the active ingredients of the present invention) and other substances. The hydrogels of the present invention release the contents when the temperature is above about 32° C. This is because the hydrogels of the present invention shrink at temperatures of about 32° C. thereby causing the discharge (release) of the absorbed substances. The rate of discharge is proportional to the rate of shrinkage of the hydrogel particle, which is dependent upon the temperature, the size of the hydrogel particle (for example, surface to size ratio and rate of temperature change affect release rate) and composition of the hydrogel (for example, crosslinking affects release rate). Hence, the release process is coupled with the hydrogel shrinking process and, therefore, the discharge rate can be controlled.

Besides the property of sustained release, the present invention provides users enhanced cooling feeling that is caused by co-effect of menthol and water contained in a hydrogel matrix.

Under the above-mentioned situation, the inventors of the present invention made an intensive study. As a result, the present inventors found that a sustained-release nasal composition which comprises a wax/petroleum jelly base, a hydrogel polymer, and invigorating, anti-fatigue compounds (menthol and menthone) can achieve the above objective. When the composition is applied on the skin, the composition comprising a hydrogel that contains a high concentration of menthol and/or menthone releases the menthol and menthone slowly thereby proving invigorating and anti-fatigue effects for at least about 1 hour and for more than about 2 hours.

The active ingredients of the present invention are menthol and menthone. The concentration of menthol and/or menthone in the composition of the present invention is from approximately 0.1% by weight to approximately 10% by weight, approximately 0.2% wt. to approximately 5% wt., approximately 0.4% wt. to approximately 2.5% wt. and approximately 0.6% wt. to approximately 2.0% wt. In another embodiment, the menthol and/or menthone that are used are components of one or more essential oils such as, for example, peppermint oil. Peppermint essential oil can contain as much as about 85% vol. menthol and/or menthone although concentrations in the 20% to 50% range are more typical. Peppermint essential oil is available commercially. See, for example, products by Plant Therapy Essential Oils, Twin Falls, Id.

The percent of hydrated hydrogel polymer in the composition of the present invention is (by weight percent) from approximately 1% to approximately 20%, approximately 4% to approximately 16%, approximately 8% to approximately 12%.

The invigorating compounds (active ingredients) used in the present invention are shown in the following formulas:

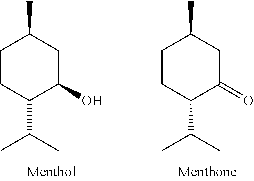

The thermo-responsive hydrogel used in the present invention includes gels made of poly(N-isopropylacrylamide) [polyNIPAM or PNIPA] (see, Park and Hoffman, 1994, J. Applied Polymer Sci., 52:85-89) its derivatives (e.g., acrylamide derivatives involve changing R groups in the formula, and its copolymer derivatives (e.g., t-butylacrylamide is one of the monomers that can be used to copolymerize polyNIPAM and shift the phase transition at lower temperature), polylactic acid (PLA) polyethylene glycol (PEG), polylactic-co-glycolic acid (PLGA), polycaprolactone (PCL) and polypeptides. The hydrogel polymers or copolymers can be used individually or in combinations of two or more.

Representative formulas are shown below:

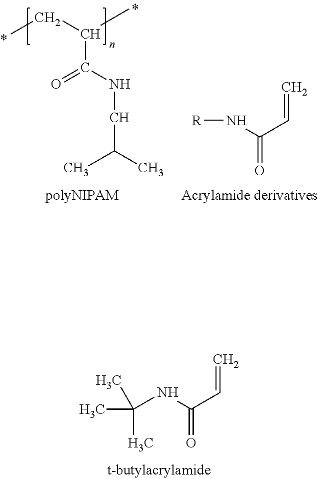

Hydrogel particles range in size from approximately 50 nm to approximately 0.5 mm. Hydrogel particles in various sizes can release liquid content at different rates and for different lengths of time and thereby contribute to the sustained-release feature of the present invention.

The hydrogel polymer may be crosslinked with crosslinking agent added in the range of 0%-approximately 10% of the dry weight of the polymer (hydrogel) used. Methylenebisacrylamide (MBA) is an example of a suitable crosslinking agent and was used in the Examples given herein. Other suitable crosslinking agents are known to those of ordinary skill in the art. In the Examples given herein, approximately 2% crosslinking agent was added to the hydrogel. The purpose of crosslinking is to build up a hydrophilic network which is suitable for containing a large amount of active ingredients and water. Although crosslinking is not essential to the present invention, its use is preferred.

For the production of an ointment, cream or balm the composition of the present invention is formulated with an ointment base. Spray formulations would not require an ointment base. The production of ointment bases is known to those of ordinary skill in the art. The ointment base used in the present invention may include one or more of petrolatum, bee's wax, plastibase, paraffin, silicone, petroleum jelly and the like. These ointment base ingredients may be used individually or in combination of two or more. The amount of the ointment base used is not particularly restricted, but falls within the range of approximately 50% -approximately 95% by weight and preferably approximately 75% -approximately 90% by weight based on the total weight of the ointment.

Menthone and menthol are used in an amount for providing invigorating benefits and relieving drowsiness symptoms, as disclosed herein. For example, the concentrations of menthone are from 0.1% to 10% and menthol from about 0.1% to 10%, each. The suitable ratio between menthone and menthol (menthone:menthol) was also determined to be appropriately 2:1 to 5:1. Other ratios have also been shown to work however with reduced effectiveness. The total amount of menthone and menthol to achieve maximum invigorating effect was determined by extensive research and was not predictable to one of ordinary skill in the art. One of ordinary skill in the art, with the aid of the teachings of the present invention, may make further refinements to the present invention without undue experimentation.

Known additives such as flavors, spices, coloring agents, surfactants and the like may be added to the sustained-release invigorating ointment according to the present invention unless the addition adversely affects the present invention. Examples include, but are not limited to, oils (e.g., coconut oil, almond oil), fragrance and colors. Water (e.g., purified) may also be included in the composition if desired or needed for solubility of components, rehydration of the hydrogel, adjustment of moisture content and/or adjustment of viscosity.

Other essential oils are known to contain menthol and menthone and are also useful in the present invention. One of ordinary skill in the art, with the guidance provided by the present specification, may incorporate one or more of these essential oils into the present invention without undue experimentation. These include, but are not limited to, Allspice, Angelica Root, Anise Star, Aphrodisiac blend, Basil, Bay, Bergamot, Birch, Black Pepper, Blood Orange, Breathe Easy blend, Cajeput, Camphor, Cananga, Caraway, Cardamom, Carrot, Cassia, Cedarwood, Chamomile (German), Chamomile (Roman), Cinnamon Bark, Cinnamon Leaf, Citronella, Clary Sage, Cleaning blend, Clementine, Clove, Cocoa Absolute, Cognac, Copaiba, Coriander, Cumin, Cypress, Cypriol, Davana, Dill Weed, Elemi, Eucalyptus, Fennel, Fir Needle, Frankincense, French Lavender, Galbanum, Geranium, Ginger, Grapefruit, Helichrysum, Ho Wood, Hydacheim, Hyssop, Invigorate blend, Jasmine Absolute, Juniper Berry, Kanuka, Key Lime, Lavender, Lemon, Lemon Eucalyptus, Lemon Myrtle, Lemongrass, Lime, Litsea, Mace, Mandarin, Manuka, Meditation blend, Melissa, Myrrh, Myrtle, Neroli, Nerolina, Niaouli, Nutmeg, Oregano, Origanum, Palmarosa, Palo Santo, Parsley, Patchouli, Penny Royal, Peppermint, Peru Balsam, Petitgrain, Pimento, Pine, Ravensara, Relaxation blend, Renew blend, Rose Absolute (Bulgarian), Rose Absolute (Moroccan), Rose Geranium, Rosemary, Rosewood, Sage, Sandalwood (Australian), Sandalwood (Indian), Sensation blend, Spearmint, Spikenard, Spruce, Stress Relief blend, Sweet Marjoram, Sweet Orange, Tagates, Tangerine, Tansy, Tarragon, Tea Tree, Thyme, Turmeric, Valerian, Vanilla, Verbena, Vetiver, Wintergreen, Yarrow and Ylang Ylang.

According to the present invention, the sustained-release invigorating composition (e.g., ointment, cream, balm, spray, etc.) of the present invention can be used by inserting it (placing it) into the nose (into the nasal cavity) or on the skin around and/or under the nose. It can also be used by the same application method for known lip balms. For example, the composition can be spread or otherwise applied to the lips. Further, the present invention can be formulated into nasal sprays by methods known to those of ordinary skill in the art and without undue experimentation. For example, reduction or elimination of the ointment base would result in a suitable nasal spray formulation.

The method for producing (formulating) the sustained-release invigorating compound of the present invention has no particular restriction. Any suitable methods known to those of skill in the art can be employed to produce the composition of the present invention and are incorporated herein by reference. For example, the ointment of the present invention can be produced by a method ordinarily used in the field or by an appropriate combination of two or more methods. Specifically, the ointment can be produced by simple mixing of individual components, e.g., a base such as an ointment base, one or more suitable hydrogels, menthol and/or menthone, water and any non-active ingredients (as indicated, supra), if desired. In one embodiment, the base is first produced, hydrated hydrogel (hydrogel and water) is added next and then peppermint oil is added. Non-active ingredients are added simultaneous with or sequential to the peppermint oil. In yet another embodiment, the menthone and menthol (either as components of peppermint oil or other essential oils or as isolated or purified chemicals) are formulated with the hydrogel (crosslinked or not crosslinked) prior to being formulated with the other ingredients of the composition.

Commercial batch mixing equipment may be utilized and the product then formed into suitable sized and shaped units simultaneously with or sequentially with packaging and instructions for use. In another method of production, the active ingredients are first mixed with the hydrogel prior to mixing with the other ingredients. In addition to being formulated as an ointment, the composition of the present invention may be formulated into a lip balm-type product. This can be achieved by, for example, increasing the amount of paraffin or wax, for example, in the base formulation. Further, the composition of the present invention may be formulated as a liquid for use as, for example, a nasal spray. Formulations are produced at room temperature and ideally below about 30° C. to limit or eliminate the release of active ingredients from the hydrogel.

### EXEMPLIFICATION

### Example 1

This example demonstrates the results of a vaporization test conducted for the sustained-release invigorating (anti-fatigue) composition of the present invention.

In order to confirm the sustained-release properties of the sustained-release invigorating composition of the present invention the following test was conducted in a temperature controllable incubator. Approximately 0.2 g of a sustained-release ointment of the present invention was spread on the surface of a plastic weighing plate. The weighing plate was put on an analytical scale in the incubator. As a control, an ointment without hydrogel (the formula is the same as sustained-release ointment except that no hydrogel was in it: in other words, the active ingredients, although present, were not incorporated in the hydrogel particles) was also tested in the same procedure as described above. The test was conducted at 35° C.±0.5° C. From the start of the test to 120 minutes the weight of ointment was monitored at intervals of 5 minutes. The results of the above test are shown in FIG. 2.

As shown in the results, the sustained-release invigorating ointment of the present invention exhibits such advantages that the menthone and menthol contained in the ointment with hydrogel is released over a long period of time. At 35° C., it took 40 minutes to release 50% of the invigorating contents in the ointment. As comparison, it took only 11 minute to release 50% percentage invigorating contents for ointment without hydrogel.

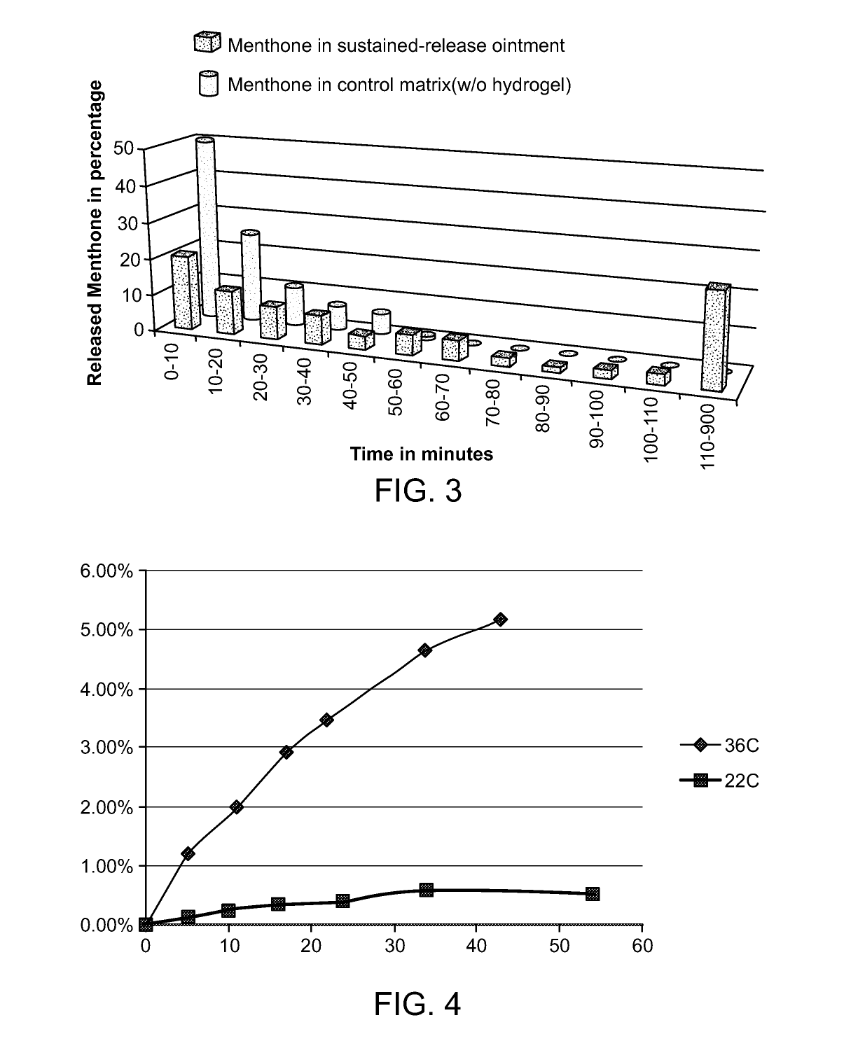

As shown in FIG. 3, in the control ointment 50% menthone were released within the first 10 minute. There was no release of menthone after 50 minutes according to this test. However, for the sustained-release ointment of the present invention, the release process (i.e., release of menthone) lasts more than 110 minutes. Even after 110 minutes there was still about 23% active ingredient reserved in the ointment.

Sample Formulation Used in the Tests:

Test formulation: ointment base (4 g), Peppermint oil (0.2 g), hydrated PNIPA hydrogel (0.4 g: 10% hydrogel, 90% water). The ointment base comprised about 20% paraffin and/or beeswax and about 80% castor oil. The wax was melted, mixed with the castor oil and cooled. This test formulation comprised approximately 0.4%-2.5% menthol/menthone. In brief, the polyNIPAM was synthesized in acetonitrile solvent in following manner: about NIPA 97%, Azobisisobutyronitrile (AIBN) about 1.5%, MBA about 2% at 65° C. for 24 hr. The synthesized PNIPAM was dialyzed, dried and grinded into white powder. The PNIPAM powder, water, menthone and menthol (constituents of the peppermint oil) were blended. The hydrated mixture was blended with an ointment base and then formed and packaged into balm stick or other packaging container.

Control formulation: ointment base (4 g), Peppermint oil (0.2 g), 360 mg H₂O (equal amount of water as contained in hydrogel in test formulation). This control formulation comprises approximately 0.4%-2.5% menthol/menthone.

### Example 2

In this example the release rate of active ingredients from the test composition (ointment) of the present invention as the result of temperature was tested.

FIG. 4 shows that the compsotion of the present invention releases active ingredients very slowly (56 ug/min at the begining 30 minutes) at 22° C. (C). The release rate was 6 times higher (359 ug/min at the beginning 30 minutes) at 36° C. (C). X-axis is time in minutes. Y-axis is release is rate of release as a percent of total weight of the test composition.

Formulation of the test sample: ointment base (4 g), Peppermint oil (0.2 g), hydrated PNIPA hydrogel (0.4 g). This test formulation comprises approximately 0.4%-2.5% menthol/menthone.

### Example 3

In this experiment the effectiveness of the composition of the present invention was tested. Two groups of subjects were used.

In Group One the ointment of the present invention was applied externally under the nose or on the upper lip. The test population was 12 adults. After application of the product they were asked following questions:

Do you feel invigorated? 11 answered yes. 1 answered not sure.

Does the product keep you awake? 8 answered yes. 4 answered not sure.

Control subjects did not report any invigoration or increased wakefulness.

In Group Two the ointment of the present invention was applied inside nose.

The test population was 8 adults. Eight of 8 felt invigorated. Eight out of 8 felt it was effective to keep them awake. Control subjects did not report any invigoration or increased wakefulness.

We claim:

1. A sustained-release composition comprising: one or more active agents selected from the group consisting of menthol and menthone, wherein said active agents are formulated in a thermo-responsive hydrogel polymer.

2. The composition of claim 1, wherein said active agents are at a concentration of about 0.4 to about 2.5 wt %.

3. The composition of claim 1, wherein said thermo-responsive hydrogel is selected from one or more of the group consisting of poly(N-isopropylacrylamide), t-butylacrylamide and acrylamide derivatives.

4. The composition of claim 1, wherein said thermo-responsive hydrogel is poly(N-isopropylacrylamide).

5. The composition of claim 1, wherein said composition additionally comprises an ointment base.

6. The composition of claim 5, wherein said ointment base comprises one or more of petrolatum, bee's wax, plastibase, paraffin, silicone and petroleum jelly.

7. The composition of claim 1, wherein said thermo-responsive hydrogel is crosslinked from 0.0% to about 10% with a crosslinking agent.

8. The composition of claim 7, wherein said crosslinking agent is methylenebisacrylamide.

9. The composition of claim 1, wherein said thermo-responsive hydrogel is comprised of particles from about 50 nm to about 0.5 mm in diameter.

10. The composition of claim 1, wherein said active agents are contained in an essential oil.

11. composition of claim 10, wherein said essential oil is peppermint essential oil.

12. composition of claim 1, wherein said composition is formulated as a spray.

13. A method of relieving fatigue in a subject, the method comprising: applying on the skin between the upper lip and nose an effective amount of the composition of claim 1.

14. A method of relieving fatigue in a subject, the method comprising: applying in the nasal cavity an effective amount of the composition of claim 1.
